(19) United States
(12) Patent Application Publication
(10) Pub. No.: US 2001/0041874 A1
(43) Pub. Date: Nov. 15, 2001

(54) BODY CANAL INTRUSION INSTRUMENTATION HAVING BIDIRECTIONAL COEFFICIENTS OF SURFACE FRICTION WITH BODY TISSUE

(76) Inventor: BORIS REYDEL, WEST CALDWELL, NJ (US)

Correspondence Address: RICHARD J. GODLEWSKI, P.O. BOX 2269, BLOOMINGTON, IN, 47402

(21) Appl. No.: 08/989,413

(22) Filed: Dec. 12, 1997

(51) Int. Cl. A61M 5/00

(52) U.S. Cl. 604/266; 604/43

(57) ABSTRACT

Medical instrumentation for intrusion of body cavities and canals, such as the gastrointestinal, urinal, vaginal and vascular tracts, is provided with unconventional surface structure replacing the conventional slippery low friction surfaces. Thus at least the surface zones of instrumentation surfaces tending to contact human tissue in ingress and egress are provided with a textured surface having a bidirectional coefficient of friction with the human tissue in the tracts being treated, which surface has the characteristic of favoring ingress movement into the work site and impeding egress movement out of the work site. The subject instrumentation with the specially textured surfaces includes viewing, therapeutic, surgical and biopsy instruments, catheters, guide cables, encompassing tubulation, and the like. This improved instrumentation expedites push endoscopy prodedures for example giving the surgeon a better feel for finding appropriate canal pathways and work sites. It is particularly advantageous in anchoring guide cables and encompassing tubulation at the work site so that the surgeon can hold them taut while passing other instruments to the work site. The textured surface also provides the advantage of feeding instrumentation into a work site by inching the distal end back and forth to pleat the canal walls behind the distal end and open up a substantially straight increment for the distal end to travel substantially parallel with the canal walls.

### TECHNICAL FIELD

This invention relates to medical instrumentation for intrusion of body cavities and canals, and more particularly it relates to surface structure on medical instruments, typically catheters, for expediting intrusion in body canals such as the gastrointestinal, urinal, vaginal and vascular tracts from exterior to the body for medical procedures.

### BACKGROUND ART

Body canal intrusion instruments are now widely used in various body canals for both examination and medical treatment of internal body tissue. Most commonly catheters are inserted for conveying fluid and instrumentation into or out of the human body. These catheters are sometimes guided along previously inserted guide wires or cables to a work site. Different types of guidewires are used to reach a desired site or to guide to that site a therapy or diagnostic device such as a catheter, dilator, balloon, stent, etc. Other instruments such as endoscopes are introduced to different systems and organs to perform diagnostic and therapeutic procedures. The art of endoscopy at present is well advanced.

In this respect, reference to the colonoscopy field or small bowel enteroscopy will identify some of the problems encountered by medical invasive techniques requiring the insertion and removal of instrumentation such as viewing, therapeutic, surgical and biopsy instruments, catheters, guide cables, encompassing tubulation, and the like. As reported at pages 120-122 of ADVANCED THERAPEUTIC ENDOSCOPY by gastroenterologist editors Jamie S. Barkin, M.D., F.A.C.P., F.A.C.G and Cesar A. O'Phelan, M.D., published by Raven Press, New York, various sorts of difficulties are encountered in the advancement of the instruments.

Lubrication of instrumentation for easier insertion is commonplace, but this causes instrumentation to egress in the presence of peristaltic waves and other bodily spasms or involuntary muscular reactions, and requires specialty anchoring techniques to prevent the instrumentation from slipping out of a critical work site location. These and other prior art problems are addressed by the present invention.

Anchoring of instrumentation at a work site by distension of instrumentation such as balloons is common in invasional medical procedures. An indwelling uretheral catheter, is known using large sized, barbed, elastically biased outwardly, rubber anchoring flaps attached at a stent distal tip for permanent retention, as disclosed in U.S. Pat. No. 3,938 529 to R. P Gibbons for INDWELLING URETERAL CATHETER, issued Feb. 17, 1976. Insertion of elastically expandable devices requires significant care and experience, and is not readily accomplished when dilation of the canal at the worksite is not desirable. Removal of medical instruments with large sized elastically-biased-open barbs can be traumatic to human tissue in the explored tracts, and do not satisfy surgeons concerned about patient comfort. Improved anchoring problems are addressed and solved by the present invention in a manner that does not significantly affect patient comfort in egress.

Among the ingress problems is the necessity to do work at an internal work site near the distal end of the invasion cathetor and/or guide wire. Because it is conventional to lubricate the cathetor, guide wire or terminal end instrument for easier insertion, it is difficult to maintain the distal end at the work site without a tendency to slip out of place, if for no other reason by a contraction of the bowels at the work site or distal end of the catheter due to a pain or spasm. The surgeon controlling the medical procedure at the work site also needs to concentrate upon that without the detractions of assuring that the instrumentation is held manually at the work site under routine procedures.

The use of conventional slippery guide wires with lubricated surfaces complicates any procedure requiring a guide wire to be held taut, for example, in order to advance a stent, catheter, dilation balloon or endoscope through a strictured area for observation and treatment of tumors, scars or cholesterol plaque. This problem is also treated by the present invention.

Accordingly the prior art techniques of invasion and removal of prior art instrumentation into body canals for medical purposes are not ideal for the purposes intended.

It is therefore a general object of this invention to improve the state of the medical arts in the procedure of inserting and withdrawing medical instrumentation to a work site in a body canal and to afford improved instrumentation structure for intrusion of body canals.

More specific objects of this invention are to solve the aforesaid problems in the prior art.

### DISCLOSURE OF THE INVENTION

This invention therefore provides improved medical instrumentation for invasion of body canals having a surface structure configured to have a bidirectional coefficient of friction with the surrounding human tissue that favors movement into the work site and impedes movement out of the work site.

This improved instrumentation relates treatment of the surfaces of catheters, guide wires and other diagnostic or therapeutic instrumentation such as viewing, therapeutic, surgical and biopsy instruments for insertion and removal by way of body canals such as gastrointestinal, urinal, vaginal and vascular tracts. Thus both plastic and metallic surfaces on such medical instrumentation that contact and move relative to human tissue in the insertion and removal process are surface treated to produce a texturized surface favoring movement along body canals into the work site and impeding movement out of the canals.

For example typical smooth instrumentation surfaces that will contact human tissue in the insertion and removal process may be surface texturized, for example, to produce angled crests of a multi-peaked or fibrous surface texture with the crests forming barb-like tips slanting at an angle toward the proximal end of a catheter, thereby to encourage the ingress of the instrumentation into the work site, and impede withdrawal. Either microscopic or macroscopic surface size may be provided, depending upon the work missions required and the characteristics of the canals to be invaded. The surface texture pattern and the geographical distribution may be custom designed for particular medical procedures. Thus, the instruments have the characteristic of grabbing or clinging to the tissue of the canal walls. In general, the textured surface may occur along the catheter length. In special cases regional surfaces may be specially provided near the work site for increasing frictional contact and affording less tendency for instrumentation to become dislodged during the course of treatment.

Thus the catheter and other medical instrumentation afforded by this invention is provided with a textured outer surface having textured structure oriented in a direction that impedes egress by increased friction with human tissue contacted along the body canal tract. Perhaps, even more importantly, this structure provides a tool for expediting insertion of the instrumentation and makes feasible improved medical methods of feeding instrumentation into the body canals.

Alternative forms of surface texture configuration may of course be adopted by those skilled in the art. It is to be expected that special purpose instruments will require different kinds of surface treatment over the range of canal sizes, the nature of the tissue-to-instrument interface and the medical process being undertaken.

This invention goes counter to the conventional catheter like invasion practice at this time which emphasises lubricated surfaces for easy entry. That produces the problem of holding the instrumentation in place at the work site during medical procedure. For example, in the face of impulsive twitches of the human tissue and the need of the operator to concentrate on the medical procedure rather than the retention of the working instrument in place during that procedure, the surface structure of this invention makes it less necessary to hold the instrumentation in place during medical procedures and reduces the possibility that the instrumentation is involuntarily dislodged from the work site.

Typically, this invention resolves a number of problems encountered when treating the gastrointestinal tract. The colon for example is curved and tortuous with sharp bends. The sigmoid part of the colon makes a sharp S-shaped turn for example. That makes it tricky to pass an endoscope from the rectum to the proximal colon through the s-shaped sigmoid. The endoscope tends to “loop out” in the sigmoid and may stall or even reverse the advancement of the tip of the endoscope.

Also, for example, to pass a catheter, guide wire or medical instrument such as an endoscope from the esophagus through the stomach toward a small intestine for treatment is simply achieved only in the esophagus region which has a straight pathway. Conversely, in order to find an exit from the stomach and to traverse the small intestines while being pushed inwardly, the process tends to form loops or coils in the catheter or guide wire. The surgeon can better “feel” when the loops being formed when the surface is textured, and may tauten a catheter or guide wire and straighten an ingress pathway by employing the frictional adherence of the instrumentation to the human canal wall tissue contacted.

With this improved “feel”, the surgeon can better thread the instrumentation along the canal pathway in a manner later discussed in some detail. Thus the surface structure on the instrumentation provided by this invention facilitates ingress to overcome some of the ingress problems above discussed.

The catheter of this invention, if inserted just in the esophagus and left alone will tend to be propulsed by peristaltic waves to the stomach and further into the small intestine because of the of the bi-directional surface texture of this invention which favors ingress and impedes egress, and thus counteracts retrogressive peristaltic waves. This is in contrast to the tendency of the instrumentation to egress in the presence of retrogressive peristaltic waves with instrumentation surface structures conventional in the prior art.

If a flexible endoscope is advanced along this pathway providing the bidirectional surface characteristics of this invention, the human tissue along the intestinal wall will tend to grab and hold on upon egress while the ingress orientation is favorably comparable with prior art lubricated instrumentation along the desired ingress route. Significantly, the bidirectionally roughened or frictional characteristics of the surface texture produces less tendency to slip the intestine wall off the instrument, and this feature is adopted in an improved method of push endoscopy, for example. Thus, an endosceope-catheter distal end may be jiggled or inch-wormed back and forth during the ingress to create pleating or accordioning of the walls the intestine tract onto the scope. Thus in the “pleating” procedure in feeding the instrumentation into the intestine, the zig-zag incremental feeding using the interface surface texture with the intestine walls moves a pleat outwardly and straigtens out the canal walls inwardly into a relatively straight increment for ingress of the instrumentation, such as the endoscope.

Thus, this invention significantly aids the surgeon in the process of feeding the instrumentation with a better “feel” for finding the intended work site in the usual push entereoscopy process described for example in the article entitled “PUSH ENTEROSCOPY” by Alexander Wilmer, MD and Paul Rutgeerts, MD, PhD., published in pages 759 to 775 of the Volume 6, Number 4, October 1996 edition of GASTROINTESTINAL ENDOSCOPY CLINICS OF NORTH AMERICA.

The intestines, particularly when being probed, are subject to involuntary peristaltic waves which propagate inwardly or retrograde outwardly. The inwardly propagating waves tend to drag the instrumentation inwardly along the canal, which aided by the surface structure of this invention retards withdrawal of the instrument by a retrograde peristaltic wave. This improves the ingress over conventional instrumentation which moves outwardly in response to the retrograde propagated waves.

The patient's comfort is also an objective of this invention, which is enhanced by simpler and less traumatic ingress methods. The bi-directional coefficient of friction which impedes the spontaneous withdrawal of the instrumentation, is not extreme enough to traumatize the canal walls, and therefore egress is not significantly more uncomfortable than with conventionally surfaced instrumentation.

Other objects, features and advantages of the invention will be found throughout the following text, drawings and claims.

### BRIEF DESCRIPTION OF THE DRAWINGS

In the accompanying drawings, wherein like reference characters refer to similar features throughout the various views:

FIG. 1 is a sketch simulating the the invasion of the vascular tract with a substantialoly hollow cylindrical catheter conveyed over a guide wire as a work instrument, which illustrates the conditions encountered in body canals by the improved instrumentation structure afforded by the present invention;

FIG. 2 is a sketch simulating the typical invasion of the gastro-intestinal tract through the esophagus with a catheter for observation and therapeutic medical procedures;

FIG. 3 is a sketch of typical catheter type medical instrumentation for invasion of the gastro-intestinal tract for observation and therapeutic procedures;

FIGS. 4 and 5 are sketches illustrating two ingress steps of the improved catheter surface configuration and advantages thereof provided in push enteroscopy ingress and egress of the gastro-intestinal tract with medical instrumentation;

FIGS. 6 and 7 are enlarged photo reproductions illustrating embodiments of applicant's unique catheter surface textures, provided on medical instruments designed for intrusion in body canals such as the gastrointestinal, genital-urinal and vascular tracts;

FIG. 8 is a fragmental sketch in section view illustrating the utility and operation of the present invention in connection with moving medical instrumentation along guide wires to a work site in a body canal;

FIG. 9 is a fragmental section sketch representing a typical medical insertion of a stent over a guide wire into a work site at a stricture tumor illustrating advantages of the invention; and

FIGS. 10A, B, C and D are fragmental section view sketches of a portion of a colon at four stages of ingress of an endoscope illustrating the improved “inch-worm” incremental back and forth method of pushing an endoscope into the small intestine afforded by this invention.

### THE PREFERRED EMBODIMENTS

This invention modifies by texturization the working surfaces of catheters, guide wires, encompassing tubulation and various working instrumentation used in diagnosing and therapeutically treating internal body tissue accessed by ingress into body canals such as the gastrointestinal, genital-urinal and vascular tracts. The surface texture that contacts body tissue in the ingress and egress of the instrumentation controlled from a location outside the body is provided with a surface texture that has bi-directional frictional characteristics to favor the ingress of the instrumentation and impede the egress.

A feature of this instrumentation is that it is far less likely to become dislodged during medical therapy or during the ingress procedure in the presence of involuntary muscular movements such as peristaltic waves. By utilization of a natural propagating peristaltic wave in the ingress of medical instrumentation, and a modified push technique made possible by the bidirectional surface friction characteristics of the instrumentation afforded by this invention, the ingress procedure is significantly enhanced and simplified. Thus, the instrument is incrementally jiggled back and forth in “inchworm” fashion during ingress in order to withdraw the textured surface while grasping canal walls thus form pleats outwardly of the distal end and straightened canal passageways inwardly of the distal end. The surface friction permits a backward jiggling movement to drag the intestine wall “accordian” like structure backward creating inwardly ahead of the “accordian” section a taut straight section of the intestine into which the distal end is then jiggled forward. Thus it is possible to straighten out bends and permit the instrumentation to be inserted step-by-step in straightened increments with less tendency to damage or penetrate the intestine walls.

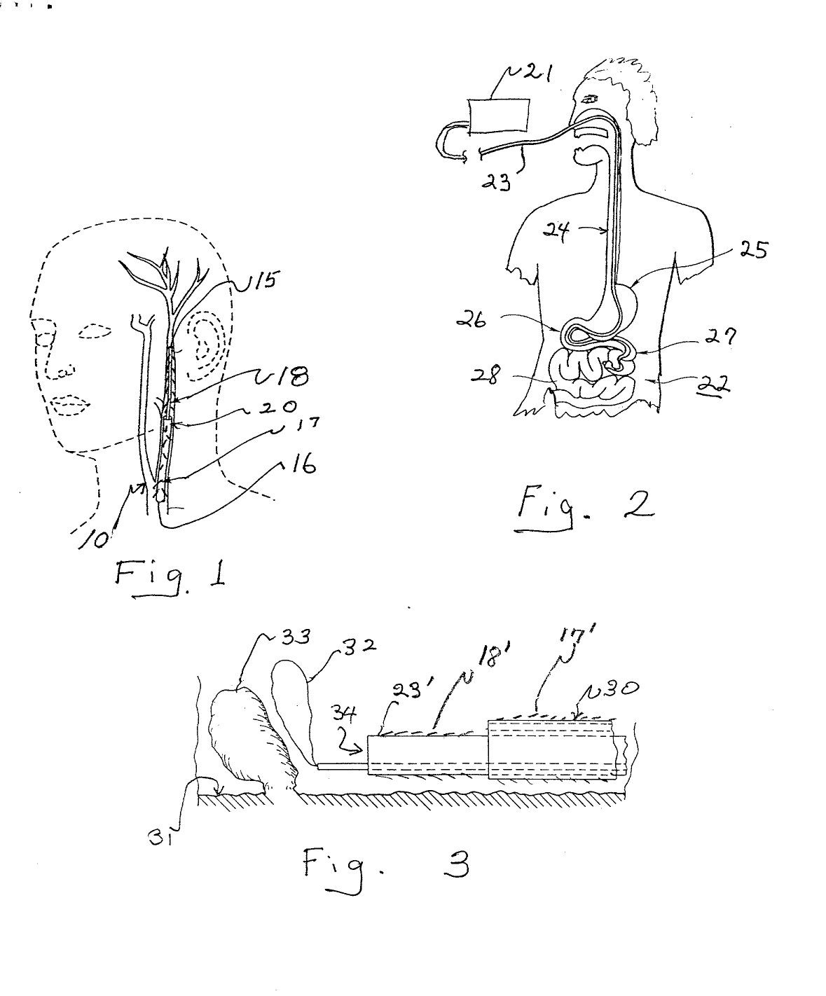

FIG. 1 represents vascular system body canals 10 receiving a guide wire 15 along which the catheter 16 is passed to a work site. In accordance with this invention, the outer surface 17 of the catheter 16 as well as the outer surface 18 of the guide wire 15 are diagrammatically shown to have textured surfaces which encounter human tissue of the canal walls upon ingress and egress into the vascular system to the work site, here designated 20. Thus, the outwardly extending lines 17, 18 represent some sort of surface texture providing a bidirectional coefficient of friction that produces the characteristic of favoring ingress and inhibiting egress respectively for both the guide wire 15 and catheter 16.

The catheter 16 is representative of other medical instrumentation and may be carrying and introducing to the work site 20 other medical instrumentation including lumens for irrigation or introducing work instruments, endoscopes for observation, balloons for enlarging the canal, and the like, all or some of which may also have the unique surface texture provided by this invention. As will be hereinafter discussed in more detail, the surface structure may be provided by various techniques on both the metal surfaces of guide wires and plastic surfaces of catheters.

FIG. 2 is a representation of the gastrointestinal tract 22, into which the catheter 23 is being introduced by way of the esophagus 24 and stomach 25. After the distal end of catheter 23 finds an exit from the stomach into the intestinal tract 26, it will then curve around intestinal loops 27 and 28, for example. For purpose of simplicity the external surface texture (17) is not shown in this view.

The ingress takes place by the well known “push endoscopy” procedure, above noted, wherein the attending surgeon from external to the body uses an implement 21 to feed the maneuverable tip of an endoscope, for example, into the intestinal tract. The surgeon by skillful manipulation during ingress avoids trauma and damage to the intestinal walls at the curves and folds that must be maneuvered, to reach a work site, or to observe the internal intestinal topography with an endoscope along the way, for example.

A typical gastrointestinal instrumentation array is shown in FIG. 3, wherein the catheter 23′ may serve as a “guide wire” for the sleeve 30 which rides over the catheter surface into the work site in the manner more fully described in my U.S. Pat. No. 5,259,366, Nov. 9, 1933 for METHOD OF USING A CATHETER-SLEEVE ASSEMBLY FOR AN ENDOSCOPE.

The simplified surface texture structure 17′, 18′ indicated is illustrative of the cylindrical surface texture's characteristic propensity to favor ingress over egress when contacting the walls 31 of the intestinal body canal. Thus, for example, if the snare 32 is used to excise the polyp 33 while viewing through the endoscope 34, and the intestinal wall involuntarily twitches or contracts, the surface texture 17′, 18′, because of its characteristic reluctance to egress will favor retention of the instrumentation in place at the work site.

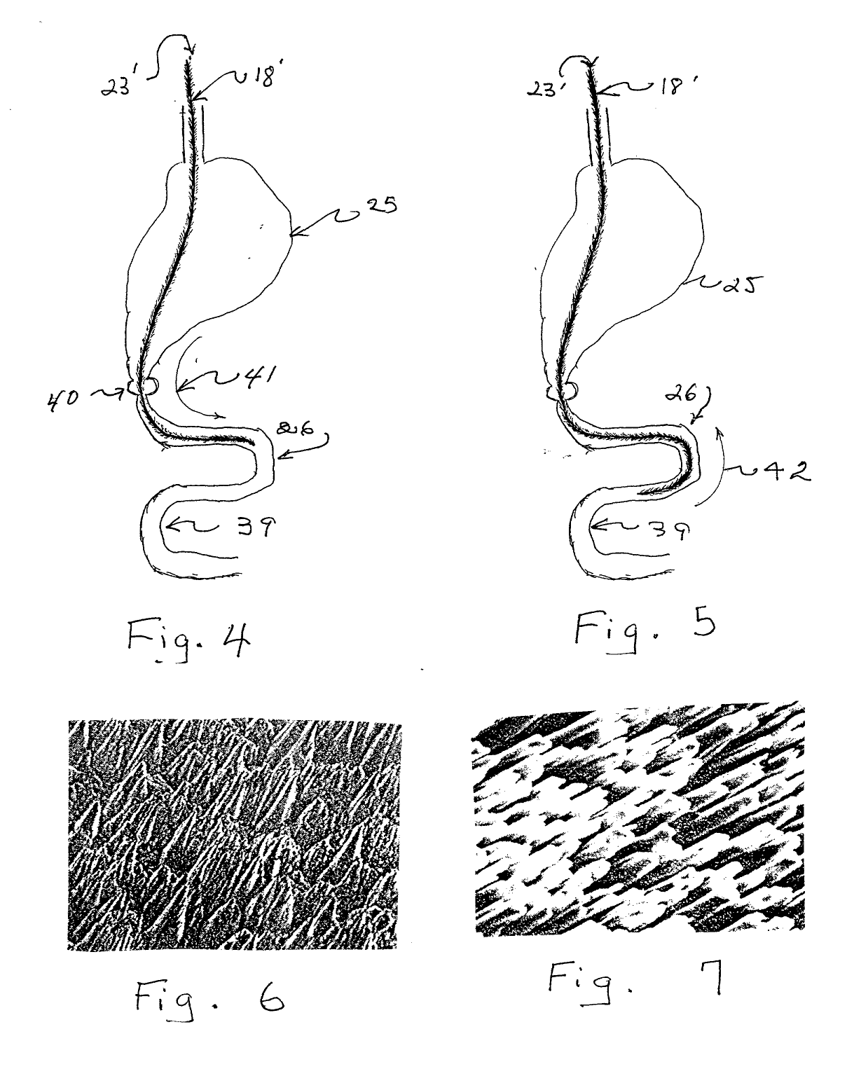

Furthermore the invention provides significant advantages in the ingress of the medical instrumentation during the push endoscopy procedure as will be explained in connection with FIGS. 4 and 5. When the distal end of the entering feeding tube catheter 23′ passes the duodenum 40 and confronts curves and folds 26 in the small intestine 39, the intestines may involuntarily generate either antegrade peristaltic waves internally directed 41, or retrograde peristaltic waves externally directed 42. The retrograde waves for example significantly impede the ingress of the catheter 23′ in the prior art with a conventional slippery surface and tends to reject the catheter in the egress direction. However, because of the frictional bi-directional surface texture 18′ of this invention, which serves as an impediment to egress when in contact with the curves and folds 26, for example, the rejection tendency is neutralized while the retrograde peristaltic waves are present. In the opposite direction, since the antegrade peristaltic waves 41 are inwardly directed, the ingress of the catheter tubing 23′ is enhanced by the peristaltic waves carrying along the catheter.

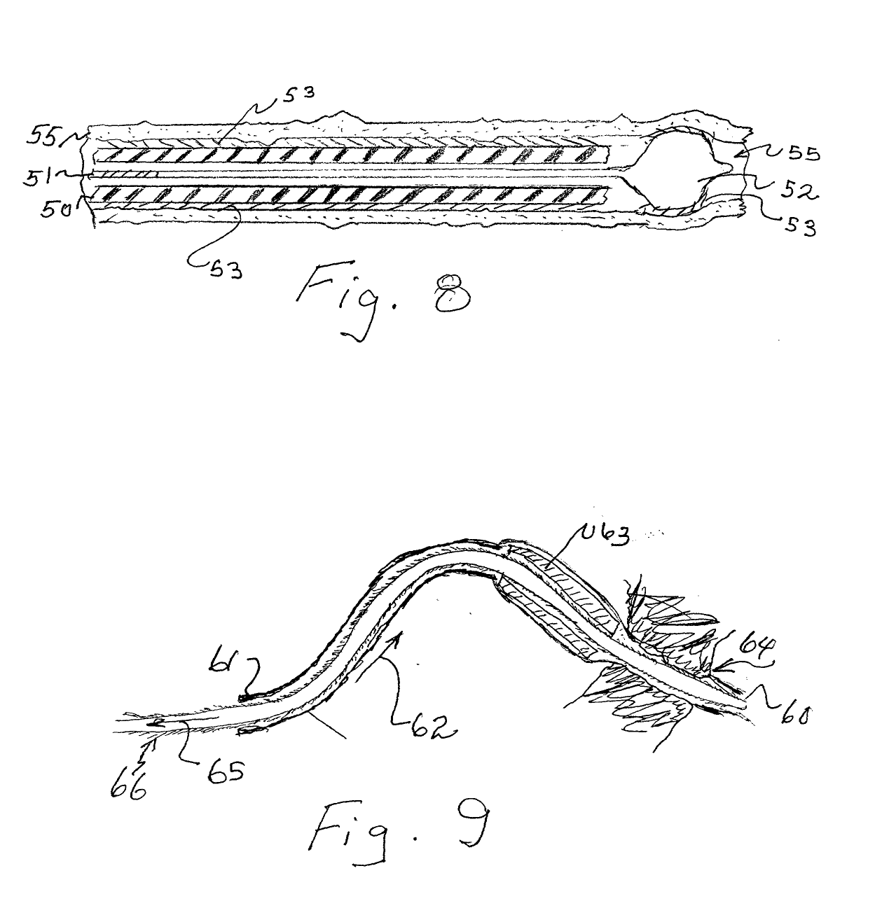

Surface texture modification of metal, polymers and ceramics for medical purposes is a well advanced art. For example, Spire Corporation, Patriots Park, Bedford Mass., which manufactures hydrophobic surfaces on catheters, also employs ion beams to produce selected surface topographies, with desired height, angle and geography on medical instrumentation surfaces. Thus, microscopic pits and pillars or other surface texture producing a bi-directional coefficient of surface friction of the type hereinbefore set forth, may be applied to desired patterns, regions or substantially the entire outer surfaces of medical instruments to be inserted into body cavities through coating techniques and ion bombardment to control surface friction and surface topography. Two examples of engraved surface geometries that meet the objectives of the present invention are illustrated in FIGS. 6 and 7. In each of these, microscopic surface peaks are angled at an appropriate slant (toward the right as shown in the drawings). The surface peaks are thus oriented to slant away from the distal end of an instrument inserted into a body canal along the surface, in resident zones that will come in contact with the canal wall tissue upon ingress, egress, and while resident at a work site.

In this manner, the surface topography meets the objectives of this invention to provide the bi-directional friction characteristic that in contact with a receptive mating surface, such as human tissue of a canal wall, favors movement in one direction and impedes movement in the opposite direction. The size, geography and surface texture characteristics can be specified to produce effective grasping characteristics on the surfaces of medical instrumentation for various medical procedures and canal characteristics. It is of course a material factor that the surface texture for insertion into body canals has favorable ingress properties without producing significant trauma or damage to the canal walls upon egress.

Thus, this invention provides for converting either plastic or metallic elongated instrumentation bodies with a smooth surface to provide the textured surface with indentations and peaks so oriented to favor ingress and impede egress of the instrumentation from body canals, at least over a significant portion of their length along invaded body canals.

At the present state of technology , other types of mechanical surface modifications can be employed and chemical processing may provide suitable grown surface topography. Also particularly with plastics, such as polyesters, fibrous surface patterns may be created by various methods, including mechanical shaping, to meet all the desired medical criteria of sanitary acceptance and surface stability that significantly eliminates the possibility of erosion, contamination or fracture in the process of therapy along the body canals.

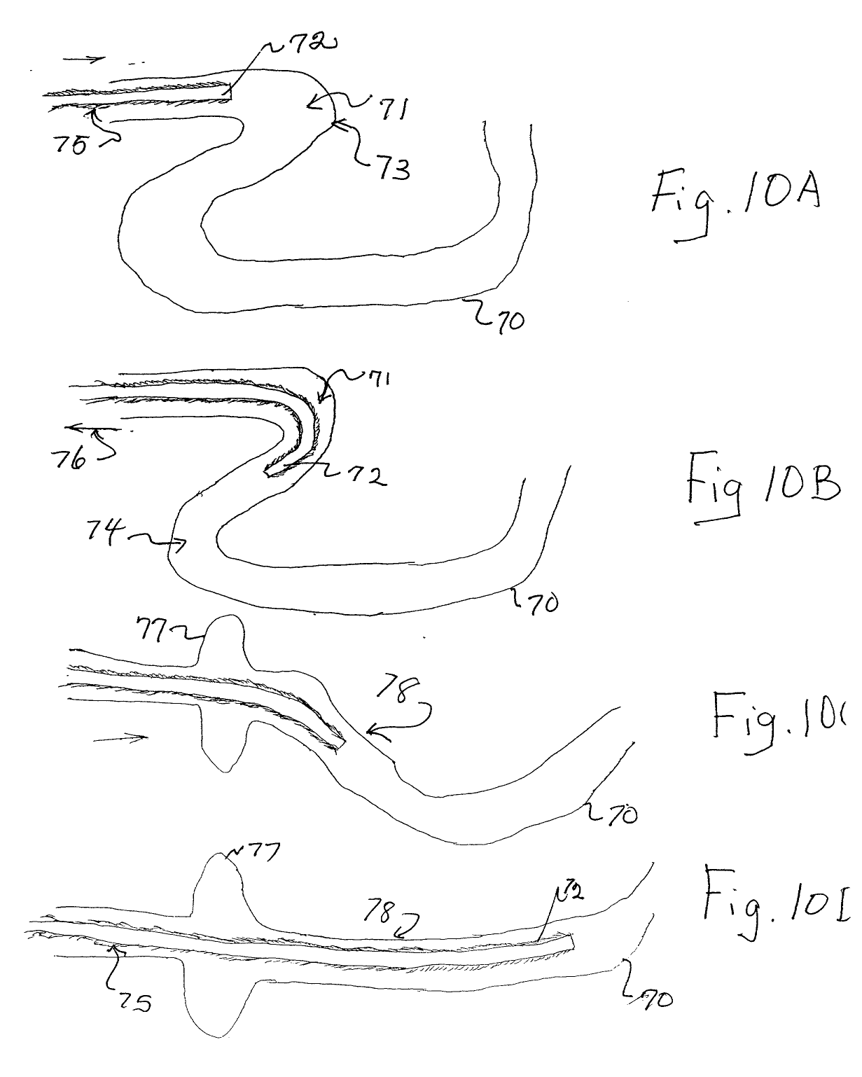

The section view sketch of FIG. 8, not to scale, represents several aspects and configurations of the invention. Thus, the plastic catheter tube 50 is seen riding upon the steel surfaced guide wire 51, which has the distal end 52 residing at a work site within the body canal 55 as anchored at least in part by the bi-directional surface texture 53 against withdrawal. This guide wire may have lumens with a shaped inflatable balloon-like distal end member 52 larger than the diameter of the guide wire for contacting amd anchoring the guide wire within the human tissue of the canal wall 52 near the distal end. Thus the balloon 52 is shown anchored for entry of the overriding catheter 50. The slanted lines 53 designate the surface texture character of the nature hereinbefore described to favor ingress and retard egress, and this extends over a substantial length of the catheter surface in this embodiment.

The catheter 50, has the surface textured (53) to produce a reluctance to egress. This structure provides a distinct advantage in the presence of therapy, where involuntary twitching or contractions, etc. might tend to dislodge the catheter from the work site at critical times when the surgeon must concentrate on a therapeutic process rather than the positioning of the catheter at the worksite, as is the case with catheters not having different bi-directional surface friction characteristics taught by this invention. Thus even without balloon or other guide wire anchors, the guide wire can be retarded from egress by a bi-directionally oriented surface texture provided by this invention.

The plastic surfaces of the catheter 50 may be treated by different processes than the steel wire 51 for texturing the respective surfaces with a desirable surface texture. For example plastics may be inscribed mechanically with outwardly slanted threads that further expedite ingress when the catheter tubing is rotated, wherein the thread inclination provides the differences in egress and ingress friction with the walls 55 of the tract under investigation. Such an embodiment would have advantages during ingress where the instrumentation may be rotated. The texturing is preferably microscopic in size so that little trauma or uncomfort is encountered by frictional interfacing with the tract wall 55 during egress. Certain variations will of course be recognized as advantageous for different medical reasons and for use in the different tracts, where catheters, guide wires and other instrumentation are in conventional use and the flexibilities, diameters and materials best suited are well known within the state of the art.

If the guide wire 51 incorporates an inflatable balloon 52 as anchoring structure, for example, that will permit the guide wire to be held quite taut from the proximal end as the catheter 50 is moved therealong to counteract any tendency to curl or coil or to loop by being dragged along with the catheter. An incremental mode of egress can be undertaken with the balloon intermittently being inflated and deflated for incremental advances to further inward locations. Also a guide wire with a bi-directional coefficient of friction with the canal wall tissue, can in the same fashion be withdrawn slightly to tauten the wire and facilitate the movement of other instrumentation over the guide wire where there is sufficient frictional anchoring to the canal walls.

In FIG. 9, the guide wire 60, having the surface texture provided by this invention that retards egress, is inserted in body canal 61 and pushed inwardly in the direction of arrow 62 for the purpose of lodging stent 63 at the work site stricture 64 constituting a stricture tumor. Since the stent 63 is larger than the strictured canal region 64 which the stent is intended to expand and support, it is difficult to insert at the work site without anchoring the guide wire at its distal end on the inward side of the stricture 64. With conventional guide wires provided with slippery surfaces for abetting ingress, the distal end of guide wire 60 tends to egress in the direction of arrow 65 and loop out below the stricture when manipulating the stent 63 to force it into the stricture 64.

However because of the textured surface 66 provided by this invention, the barbs still favor ingress, but will tend to grab the surrounding canal wall tissue and prevent the egress, particularly at and beyond the stricture 64. Thus, the guide wire 60 may be tautened with the distal end in place at the work site to guide the stent into the stricture 64.

In FIG. 10, a sigmoid section of colon 70 is represented with a curved path 71, which gives problems of ingress of the endoscope catheter array 72. Normally the distal end of the catheter 72 would have a tendency to snag or embed into the curved wall 73 rather than to bend about the curved path 71 sketched in FIG. 10B and progress around the next curve 74. For this reason egress must be skillfully and carefully achieved by the surgeon. However with the textured surface 75 which impedes egress because of friction with the wall tissue of colon 70 the “barbs” contact the walls during egress as represented by arrow 76. Thus the endoscope catheter surface structure will progress outwardly from the position inwardly of curve 71 in FIG. 10B to configure the colon into the position in FIG. 10C as the catheter is tautened by incrementally inching it outwardly. This drags the colon walls back to form the pleat 77 and tends to straighten out the curves in the colon inwardly in the region 78. Then as shown in Figure lOD, the catheter 72 moves directly along the straightend out colon region 78 with far less tendency to encounter the sidewalls and cause discomfort or damage.

Thus, the catheter textured surface significantly aids the ingress of the medical instrumentation into curved paths of body canals and permits faster and safer ingress by the employment of a “jiggling” or “inchworming” ingress method where the catheter is incrementally jiggled back and forth, thereby tautening as the textured surface 75 grabs the canal walls in the egress increment and in unimpeded fashion progresses into the “straightened” out canal increment resulting from the tautening.

Thus it is seen that the unique instrumentation surface texture afforded by this invention facilitates ingress of medical instrumentation into body canals so that the instruments can be used for observation and therapy at an internal work site, and serves advantageously to feed the instruments into the work site and to improve their retention or lodging function at the work site during medical procedures so that they are less apt to be inadvertently moved away from an active work site as likely could happen more frequently with conventional surface structure.

Further advantages of the invention are providing in guiding an elongated accessory conveying medical instrument of the type for observation and therapeutic medical work into an internal work site in a body canal. The bi-directional coefficient of friction against the canal wall tissue afforded by this invention is used advantageously as a gripping medium on the instrumentation surface. Thus a guide wire can be held taut against the frictional grip into the human tissue afforded by the surface texture while other medical instrumentation is carried along to the work site vicinity.

Accordingly the state of the art is advanced, and those features of novelty indicative of the spirit and nature of the invention are set forth with particularity in the following claims.

1. An instrumentation entry system for insertion from outside a body into a work site in a gastrointestinal tract for resolving entry, movement and retention problems caused by interfaces with inserted devices and the tissue of the gastrointestinal tract, comprising in combination:
entry means for insertion into the gastrointestinal tract, movement along the tract and positioning at a resident work site, said entry means being longitudinal and semi-flexible having a length extending from outside the body to the work site with a propensity to move in a straight line and to flex around curved paths in the gastrointestinal tract, and textured surface structure on said entry means disposed to engage tissue along the gastrointestinal tract as the distal end moves into and out of the work site and resides at the work site, said textured surface having a bi-directional coefficient of friction with human tissue engaged by the textured surface structure in the gastrointestinal tract that favors movement toward the work site and impedes witdrawal movement out of the work site.

2. The entry system of claim 1 wherein the entry means further comprises a catheter tubing.

3. The entry system of claim 1 wherein the entry means further comprises a guide cable system for conveying instrumentation into the work site.

4. The entry system of claim 1 wherein the entry means further comprises endoscopic instrumentation.

5. The entry system of claim 1 wherein the entry means has a substantially cylindrical cross section.

6. The entry system of claim 1 wherein the textured surface structure comprises a surface topography pattern defining surface peaks slanted in a direction away from a distal end of the entry means when situated in a body canal.

7. A longitudinal catheter of a length reaching from outside a body into a work site adapted for entry into, movement along and withdrawal from a body canal for supporting medical surveillance and therapy at a resident work site inside the body, comprising in combination:
a hollow flexible tubing with an external surface structure disposed to engage human tissue within said canal during movement into and out of said work site and in residence at the work site,
said surface structure having a texture disposed in a designated pattern along a significant portion of its length to produce a bidirectional coefficient of friction with engaged tissue within said canal that favors entry into the work site and impedes movement out of the work site.

8. Instrumentation for entry into and along a body tissue canal and movement into a resident work site inside a body, comprising in combination:
an elongated guide cable with an external surface structure disposed to engage body tissue along said canal during movement toward and away from said work site and in residence at the work site, said surface structure being textured along a significant portion of its length to provide a bidirectional coefficient of friction with engaged tissue within said canal that favors entry into the work site and impedes movement out of the work site.

9. Instrumentation for entry into and along a body tissue canal for movement into a resident work site inside a body, comprising in combination
medical instrumentation movable into said work site by an attached longitudinal body extending from outside the body, and external surface structure on said instrumentation disposed to engage tissue within said canal during movement into and out of said work site and in residence at the work site,
said external surface structure at least in a designated region along the length of said longitudinal body being textured to produce a bidirectional coefficient of friction with engaged tissue within said canal that favors entry into the work site and impedes movement out of the work site.

10. The method of treating surgical instrumentation introduced by an elongated body adapted to enter an internal body work site from a position outside the body for viewing and therapy of human tissue along body canals, typically the gastrointestinal, urinal, vaginal and vascular tracts, comprising the steps of:
identifying at least one surface structure zone of the instrumentation outer surface that engages and interacts with human tissue along canal walls upon ingress and egress to and from the work site, and
providing a textured surface on the instrumentation at said zone having a bidirectional coefficient of friction with the human tissue with the characteristic that favors ingress of the instrumentation into said canals and impedes egress.

11. The method of claim 10 further comprising the step of providing said textured surface by ionization bombardment.

12. The method of claim 10 further comprising the step of providing said textured surface by processing a smooth surface to produce a microtexture configuration having said bidirectional coefficient of friction characteristic.

13. The surgical method of invading body canals, typically the gastrointestinal, urinal, vaginal and vascular tracts, with instrumentation for medical observation and therapeutic treatment at selected work sites, comprising the steps of:
providing instrumentation with a surface texture having a bidirectional coefficient of friction with human tissue engaged in ingress and egress of the instrumentation into said tracts, said surface texture having the characteristic that favors ingress and impedes egress, and
moving the instrumentation into a worksite along one of said tracts.

14. The method of medical observation and therapy at a work site within a body canal, comprising the steps of:
feeding flexible conveying guide means with an external surface textured to favor ingress and impede egress at least in the vicinity of a distal end from outside the body into the work site for guiding observation and therapeutic work instruments thereover into the work site,
holding the guide means taut as a function of friction between the human tissue and the surface texture of the guide means for expediting the conveyance of the work instruments to the work site, and
conveying a work instrument along the guide means to the vicinity of the work site while the guide means is held taut.

15. The method of medically intruding within one of the body tracts of the group including the gastrointestinal, urinal, vaginal and vascular tracts comprising the steps of:
providing a catheter with a textured outer surface provided with directional surface structure oriented to impede egress by increased friction with human tissue contacted along the tract and to favor ingress, and
inserting said catheter into a work site along the selected tract by inching a distal end of the catheter back and forth while the textured surface engages tract tissue walls in a manner pleating the wall tissue behind the distal end and straightening out tract walls ahead of the distal end, thereby to feed the catheter toward the work site substantially by incremental advancement bites substantially directed inwardly parallel to the tract walls.
